(19) United States
(12) Patent Application Publication
(10) Pub. No.: US 2022/0017543 A1
(43) Pub. Date: Jan. 20, 2022

(54) METHOD FOR PREPARING BORIC ACID ESTER USING UNCATALYZED HYDROBORATION OF CARBOXYLIC ACID

(71) Applicant: SOOCHOW UNIVERSITY, Suzhou (CN)

(72) Inventors: Mingqiang XUE, Suzhou (CN); Xiaojuan XU, Suzhou (CN); Dandan YAN, Suzhou (CN)

(21) Appl. No.: 17/311,314

(22) PCT Filed: Mar. 7, 2019
(86) PCT No.: PCT/CN2019/077387

§ 371 Date: Jun. 4, 2021

(30) Foreign Application Priority Data
Dec. 6, 2018 (CN) 201811489596.0
Dec. 6, 2018 (CN) 201811490360.9

(51) Int. Cl. C07F 5/04; C07C 29/09

(52) U.S. Cl. CPC C07F 5/04; C07C 29/095

(57) ABSTRACT

Disclosed is a method for preparing a boric acid ester using non-catalyzed hydroboration of a carboxylic acid. The method includes: in an inert gas atmosphere, mixing pinacolborane and a carboxylic acid and stirring until uniform in a reaction flask subjected to dehydration and deoxygenation treatments, reacting for 6-12 hours to obtain the boric acid ester, then adding silica gel and methanol, and conducting a hydrolysis reaction to prepare an alcohol compound. The carboxylic acid is acetic acid, caproic acid, pentanoic acid, heptanoic acid, trimethylacetic acid, adipic acid, benzoic acid, 4-bromobenzoic acid, 4-fluorobenzoic acid, 1-naphthoic acid, 2-methoxybenzoic acid, 4-tert-butylbenzoic acid, 4-ethoxybenzoic acid, 2-bromobenzoic acid, 4-iodobenzoic acid, 3-phenylpropionic acid, diphenyl acetic acid, 2-phenylbutyric acid, indole-3-acetic acid, o-carboxyl phenylacetic acid or 2-methyl-5-bromobenzoic acid. The present invention utilizes a carboxylic acid to efficiently undergo hydroboration with borane without a catalyst for the first time.

### TECHNICAL FIELD

The invention relates to the application field of green chemistry, particularly relates to a method for preparing the borate ester by non-catalytic hydroboration with carboxylic acid.

### BACKGROUND ART

Organic boronic acid esters can be regarded as derivatives in which the hydrogen in orthoboric acid B(OH)₃ is replaced by organic groups, in addition to metaborate (ROBO)₃. Due to its stability and low toxicity, borate is widely used in various fields. It is a main raw material for the synthesis of boron-containing compounds. Borate compounds can be used not only as rust inhibitors, preservatives, polymer additives, anti-wear additives, automobile brake fluids, gasoline additives, flame retardants, but also as lubricant additives.

The reduction catalyst system reported in the literature for the synthesis of borate esters is mainly the carboxylic acid hydroboration catalyzed by LiAlH₄ and NaBH₄, SmI₂—H₂O-Et₃N and various transition metal complex catalysts. However, these methods have obvious shortcomings:LiAlH₄ and NaBH₄ systems have great safety risks, the SmI₂—H₂O-Et₃N system requires a lot of excess reagents, and the Ru, Rh, Ir, Co transition metal complex systems require high temperature and high pressure. The difference between the nucleophilic addition reaction activity of carboxylic acid and aldehyde and ketone: (1) The active hydrogen of carboxylic acid is easy to leave, so its two O's are actually equivalent, so from the perspective of steric structure, the steric hindrance of carboxyl group is more Large, and the existence of carboxyl hydrogen bonds makes the electron cloud density of the entire carbonyl group larger, and it is more difficult for nucleophiles to attack the active center; (2) Nucleophiles attack the carbonyl carbon first, which is related to the electron cloud density on the carbon. Small advanced attacks, such as aldehydes and ketones have a smaller electron cloud density than esters and amides, so they are highly active and will react preferentially. Those with large steric effects are not easy to react; (3) When forming a transition state, it depends on the leaving group, aldehyde The leaving groups of ketones are alkyl and hydrogen, both of which are not easy to leave. Therefore, aldehydes and ketones only undergo addition without elimination, which is different from carboxylic acids and their derivatives. On the one hand, the existing methods use catalysts that are difficult to synthesize and cost high; on the other hand, the catalytic reaction requires a reaction temperature of 60° C. and a reaction time of 24 hours.

### Technical Problem

This invention is in order to supply a method that conforms to the principles of green chemistry. Without solvent and catalyst, the borate ester is prepared by hydroboration with carboxylic acid and pinacol borane, generally, product alcohol. The method is environmentally friendly and has a good substrate application range.

### Technical Solution

In order to achieve the above purposes, the technical proposal adopted by the invention is:

A method for preparing the borate ester by non-catalytic hydroboration with carboxylic acid, the method comprising the following steps: without solvent and catalyst, the borate ester is prepared by non-catalytic hydroboration with carboxylic acid and borane.

A method for preparing alcohol compound with carboxylic acid, the method comprising the following steps: without solvent and catalyst, the borate ester is prepared by non-catalytic hydroboration with carboxylic acid and borane; after the hydroboration, add silica gel, methanol, and the alcohol compound is prepared by hydrolysis reaction.

The application of preparation of borate or alcohol compounds with carboxylic acid and borane, wherein carried out without a catalyst.

In the above, the borane is pinacol borane; the carboxylic acid is acetic acid, caproic acid, valeric acid, heptanoic acid, trimethylacetic acid, adipic acid, benzoic acid, 4-bromo-benzoic acid, 4-fluorobenzoic acid, 1-naphthoic acid, 2-methoxybenzoic acid, 4-tert-butylbenzoic acid, 4-ethyl oxybenzoic acid, 2-bromobenzoic acid, 4-iodobenzoic acid, 3-phenylpropionic acid, diphenylacetic acid, 2-phenylbutyric acid, indole-3-acetic acid, o-carboxyphenylacetic acid or 2-methyl 5-Bromobenzoic acid

In the above, the molar ratio of the carboxylic acid and borane is from 1:3 to 1:7.

In the above, the hydroboration is carried at room temperature for 6 to 12 hours; preferably, if the carboxylic acid is acetic acid, caproic acid, valeric acid, heptanoic acid, trimethylacetic acid, or adipic acid, the hydroboration spends from 8 to 10 h.

In the present invention, the conditions of the hydrolysis reaction are at 50° C. for 3 h; the amount ratio of carboxylic acid to silica gel and methanol is 1 mmoL:2 g:5 mL-6 mL; preferably, when the carboxylic acid is acetic acid, caproic acid, valeric acid, heptanoic acid, Trimethyl acetic acid or adipic acid, the dosage ratio of carboxylic acid to silica gel and methanol is 1 mmoL:2 g:5 mL, when the carboxylic acid is benzoic acid, 4-bromobenzoic acid, 4-fluorobenzoic acid, 1-naphthoic acid, 2-methoxybenzoic acid, 4-tert-butylbenzoic acid, 4-ethoxybenzoic acid, 2-bromobenzoic acid, 4-iodobenzoic acid, 3-phenylpropionic acid, diphenylacetic acid, 2- In the case of phenylbutyric acid, indole-3-acetic acid, o-carboxyphenylacetic acid or 2-methyl-5-bromobenzoic acid, the dosage ratio of carboxylic acid to silica gel and methanol is 1 mmoL:2 g:6 mL

In the above technical scheme, the specific steps of the method for preparing borate ester by hydroboration with carboxylic acid is:

At the inert gas, uniformly stirring and mixing carboxylic acid and borane for 6 to 12 h; after that, the reaction is stopped by contacting air to obtain borate.

The specific steps of the method for preparing alcohol compound with carboxylic acid are:

Under the inert gas, uniformly stirring and mixing carboxylic acid and borane for 6 to 12 h; the reaction is stopped by contacting air to obtain borate, and then add silica gel, methanol at 50° C. for 3 h, after hydrolysis reaction, the product alcohol compound is extracted three times with ethyl acetate, and the organic layers were combined, dried with anhydrous sodium sulfate, the solvent was removed under reduced pressure, and purified by silica gel (100-200 mesh) column chromatography, washed with ethyl acetate/hexane (1:5 volume ratio) mixture.

### Beneficial Effect

Due to the application of the above technical solutions, the present invention has the following advantages compared with the prior art:

1. The invention can carry out the hydroboration reaction of carboxylic acid and pinacol borane without solvent and catalyst for the first time, thereby developing an efficient and green method for preparing alkyl borate.

2. The invention can carry out the hydroboration reaction of carboxylic acid and borane with high activity at room temperature. The reaction can be carried out for 6-12 hours, and the conversion rate can reach more than 90%. Compared with the existing catalytic system, without solvent and catalyst, the reaction can reach a high conversion rate.

3. The solvent-free catalyst-free carboxylic acid hydroboration disclosed in the present invention has a wide range of application to substrates, is suitable for carboxylic acids with different substituent positions and different electronic effects, and provides more options for the industrial synthesis of borate esters. The process is simple and controllable, the yield is high, the post-processing of the product is easy, and it is suitable for industrial production.

### DESCRIPTION OF THE DRAWINGS

FIG. 1 is the schematic diagram of the reaction in Example 1;

FIG. 2 is the schematic diagram of the reaction in Example 18.

### EMBODIMENTS OF THE INVENTION

The following further describes the present invention with reference to the embodiments:

### Example 1: Borate Ester Prepared from Pinacolborane and Benzoic Acid with a Molar Ratio of 3:1

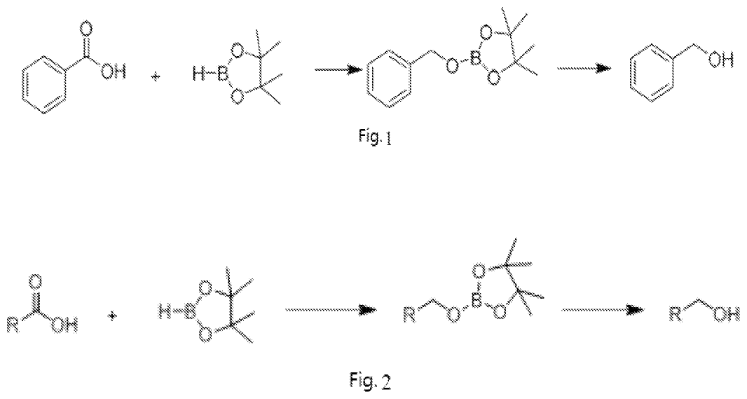

Under the protection of N₂, loading the benzoic acid (61.1 mg, 0.5 mmol) to the reaction bottle, which was treated by dehydration and deoxidation, then adding a pinacolborane (218 μL, 1.5 mmol) with pipette gun, after reacting for 12 h at room temperature, the reaction was removed from the glove box, the reaction was terminated, and the borate was obtained. The mes-trimethoxybenzene (84.15 mg, 0.5 mmol) was used as the internal standard. The stock solution was dissolved in CDCl₃ stirring for 10 minutes, sampled, and equipped with nuclear magnetism. After calculation, conversion rate of 1H is 99%, and the reaction diagram is shown in FIG. 1. 1H NMR analysis of the product is: 1H NMR (400 MHz, CDCl₃): δ 7.22-7.32 (m, 5H, ArH), 4.92 (s, 2H, CH₂), 1.26 (s, 36H, CH₃). The boric acid ester was further hydrolyzed into alcohol, adding 1 g silica gel with 3 mL methanol as the solvent at 50° C. for 3 h. After the reaction, the product pure primary alcohol was extracted three times with ethyl acetate, and the organic layers were combined, dried with anhydrous sodium sulfate, the solvent was removed under reduced pressure, and purified by silica gel (100-200 mesh) column chromatography, washed with ethyl acetate/hexane (1:5 volume ratio) mixture, with a separation yield of 92%. For the reaction diagram, see the FIG. 1. 1H NMR analysis of the product is: 1H NMR (400 MHz, CDCl₃): δ 7.22-7.29 (m, 5H, ArH), 4.61 (s, 2H, CH₂), 1.87 (br s, 1H, OH).

### Example 2: Borate Ester Prepared from Pinacolborane and Benzoic Acid with a Molar Ratio of 4:1

Under the protection of N₂, loading the benzoic acid (60.3 mg, 0.5 mmol) to the reaction bottle, which was treated by dehydration and deoxidation, then adding a pinacolborane (289 μL, 2 mmol) with pipette gun, after reacting for 6 h at room temperature, the reaction was removed from the glove box. The mes-trimethoxybenzene (83.05 mg, 0.5 mmol) was used as the internal standard. The stock solution was dissolved in CDCl₃ stirring for 10 minutes, sampled, and equipped with nuclear magnetism. After calculation, conversion rate of 1H was 93%. Continuing hydrolyzing to obtain pure primary alcohol with an isolated yield of 85%.

### Example 3: Borate Ester Prepared from Pinacolborane and Benzoic Acid with a Molar Ratio of 4:1

Under the protection of N₂, loading the benzoic acid (59.9 mg, 0.5 mmol) to the reaction bottle, which is treated by dehydration and deoxidation, then adding a pinacolborane (289 μL, 2 mmol) with pipette gun, after reacting for 12 h at room temperature, the reaction was removed from the glove box. The mes-trimethoxybenzene (82.50 mg, 0.5 mmol) was used as the internal standard. The stock solution was dissolved in CDCl₃ stirring for 10 minutes, sampled, and equipped with nuclear magnetism. After calculation, conversion rate of 1H was 99%. Continuing hydrolyzing to obtain pure primary alcohol with an isolated yield of 92%.

### Example 4: Borate Ester Prepared from Pinacolborane and Benzoic Acid with a Molar Ratio of 5:1

Under the protection of N₂, loading the benzoic acid (60.8 mg, 0.5 mmol) to the reaction bottle, which is treated by dehydration and deoxidation, then adding a pinacolborane (362 μL, 2.5 mmol) with pipette gun, after reacting for 9 h at room temperature, the reaction was removed from the glove box. The mes-trimethoxybenzene (83.74 mg, 0.5 mmol) was used as the internal standard. The stock solution was dissolved in CDCl₃ stirring for 10 minutes, sampled, and equipped with nuclear magnetism. After calculation, conversion rate of 1H was 99%. Continuing hydrolyzing to obtain pure primary alcohol with an isolated yield of 92%. And add 0.5 mL of anhydrous 1,4-dioxane to the reaction system, the product yield was 18%.

### Example 5: Borate Ester Prepared from Pinacolborane and 4-Fluorobenzoic Acid with a Molar Ratio of 4:1

Under the protection of N₂, loading the 4-fluorobenzoic acid (70.8 mg, 0.5 mmol) to the reaction bottle, which is treated by dehydration and deoxidation, then adding a pinacolborane (290 μL, 2 mmol) with pipette gun, after reacting for 12 h at room temperature, the reaction was removed from the glove box, the reaction was terminated, and the borate was obtained. The mes-trimethoxybenzene (84.99 mg, 0.5 mmol) was used as the internal standard. The stock solution was dissolved in CDCl₃ stirring for 10 minutes, sampled, and equipped with nuclear magnetism. After calculation, conversion rate of 1H was 90%. 1H NMR analysis of the product is: 1H NMR (400 MHz, CDCl₃): δ 7.22 (br s, 2H, ArCH), 6.92 (t, 2H, ArCH), 4.76 (s, 2H, OCH₂), 1.26 (s, 36H, CH₃). The boric acid ester was further hydrolyzed into alcohol, add 1 g silica gel with 3 mL methanol as the solvent at 50° C. for 3 h. After the reaction, the product pure primary alcohol was extracted three times with ethyl acetate, and the organic layers were combined, dried with anhydrous sodium sulfate, the solvent was removed under reduced pressure, and purified by silica gel (100-200 mesh) column chromatography, washed with ethyl acetate/hexane (1:5 volume ratio) mixture, with a separation yield of 90%. ¹H NMR analysis of the product is: ¹H HMR (400 MHz, CDCl₃): δ 7.24 (br s, 2H, ArCH), 6.98 (t, 2H, ArCH), 4.56 (s, 2H, CH₂), 2.27 (br s, 1H, OH).

### Example 6: Borate Ester Prepared from Pinacolborane and 4-Bromo-Benzoic Acid with a Molar Ratio of 4:1

Under the protection of N₂, loading the 4-bromo-benzoic acid (100 mg, 0.5 mmol) to the reaction bottle, which is treated by dehydration and deoxidation, then adding a pinacolborane (289 μL, 2 mmol) with pipette gun, after reacting for 12 h at room temperature, the reaction was removed from the glove box, the reaction was terminated, and the borate was obtained. The mes-trimethoxybenzene (83.67 mg, 0.5 mmol) was used as the internal standard. The stock solution was dissolved in CDCl₃ stirring for 10 minutes, sampled, and equipped with nuclear magnetism. After calculation, conversion rate of 1H is 95%. 1H NMR analysis of the product is: 1H NMR (400 MHz, CDCl₃): δ 7.41 (br s, 2H, ArCH), 7.18 (t, 2H, ArCH), 4.82 (s, 2H, OCH₂), 1.21 (s, 36H, CH₃). The boric acid ester was further hydrolyzed into alcohol, add 1 g silica gel with 3 mL methanol as the solvent at 50° C. for 3 h. After the reaction, the product pure primary alcohol was extracted three times with ethyl acetate, and the organic layers were combined, dried with anhydrous sodium sulfate, the solvent was removed under reduced pressure, and purified by silica gel (100-200 mesh) column chromatography, washed with ethyl acetate/hexane (1:5 volume ratio) mixture, with a separation yield of 93%. 1H NMR analysis of the product is: 1H NMR (400 MHz, CDCl₃): δ 7.41 (br s, 2H, ArCH), 7.19 (t, 2H, ArCH), 4.60 (s, 2H, CH₂), 2.26 (br s, 1H, OH).

### Example 7: Borate Ester Prepared from Pinacolborane and 2-Methoxybenzoic Acid with a Molar Ratio of 4:1

Under the protection of N₂, loading the 2-methoxybenzoic acid (76.2 mg, 0.5 mmol) to the reaction bottle, which is treated by dehydration and deoxidation, then adding a pinacolborane (290 μL, 2 mmol) with pipette gun, after reacting for 12 h at room temperature, the reaction was removed from the glove box, the reaction was terminated, and the borate was obtained. The mes-trimethoxybenzene (84.23 mg, 0.5 mmol) was used as the internal standard. The stock solution was dissolved in CDCl₃ stirring for 10 minutes, sampled, and equipped with nuclear magnetism. After calculation, conversion rate of 1H is 99%. 1H NMR analysis of the product is: 1H NMR (400 MHz, CDCl₃): δ 7.42 (d, 1H, ArCH), 7.23 (t, 1H, ArCH), 6.96 (t, 1H, ArCH), 6.84 (d, 1H, ArCH), 4.98 (s, 2H, OCH₂), 1.27 (s, 36H, CH3). The boric acid ester was further hydrolyzed into alcohol, add 1 g silica gel with 3 mL methanol as the solvent at 50° C. for 3 h. After the reaction, the product pure primary alcohol was extracted three times with ethyl acetate, and the organic layers were combined, dried with anhydrous sodium sulfate, the solvent was removed under reduced pressure, and purified by silica gel (100-200 mesh) column chromatography, washed with ethyl acetate/hexane (1:5 volume ratio) mixture, with a separation yield of 90%. 1H NMR analysis of the product is: 1H NMR (400 MHz, CDCl₃): δ 7.42 (d, 1H, ArCH), 7.23 (t, 1H, ArCH), 6.96 (t, 1H, ArCH), 6.84 (d, 1H, ArCH), 4.69 (s, 2H, CH2), 3.87 (br s, 1H, OH), 1.23 (s, 3H, CH3).

### Example 8: Borate Ester Prepared from Pinacolborane and 1-Naphthoic Acid with a Molar Ratio of 4:1

Under the protection of N₂, loading the 1-naphthoic acid (85.4 mg, 0.5 mmol) to the reaction bottle, which is treated by dehydration and deoxidation, then adding a pinacolborane (289 μL, 2 mmol) with pipette gun, after reacting for 12 h at room temperature, the reaction was removed from the glove box, the reaction was terminated, and the borate was obtained. The mes-trimethoxybenzene (83.42 mg, 0.5 mmol) was used as the internal standard. The stock solution was dissolved in CDCl₃ stirring for 10 minutes, sampled, and equipped with nuclear magnetism. After calculation, conversion rate of 1H is 91%. 1H NMR analysis of the product is: 1H NMR (400 MHz, CDCl₃): δ 8.02 (d, 1H, ArCH), 7.80-7.82 (m, 2H, ArCH), 7.75 (d, 1H, ArCH), 7.38-7.48 (m, 3H, ArCH), 5.37 (s, 2H, OCH₂), 1.23 (s, 36H, CH₃). The boric acid ester was further hydrolyzed into alcohol, add 1 g silica gel with 3 mL methanol as the solvent at 50° C. for 3 h. After the reaction, the product pure primary alcohol was extracted three times with ethyl acetate, and the organic layers were combined, dried with anhydrous sodium sulfate, the solvent was removed under reduced pressure, and purified by silica gel (100-200 mesh) column chromatography, washed with ethyl acetate/hexane (1:5 volume ratio) mixture, with a separation yield of 91%. 1H NMR analysis of the product is: 1H NMR (400 MHz, CDCl₃): δ 8.02 (d, 1H, ArCH), 7.80-7.82 (m, 2H, ArCH), 7.73 (d, 1H, ArCH), 7.38-7.48 (m, 3H, ArCH), 5.01 (s, 2H, CH2), 2.31 (br s, 1H, OH).

### Example 9: Borate Ester Prepared from Pinacolborane and 4-Tert-Butylbenzoic Acid with a Molar Ratio of 4:1

Under the protection of N₂, loading the 4-tert-butylbenzoic acid (88.9 mg, 0.5 mmol) to the reaction bottle, which is treated by dehydration and deoxidation, then adding a pinacolborane (290 μL, 2 mmol) with pipette gun, after reacting for 12 h at room temperature, the reaction was removed from the glove box, the reaction was terminated, and the borate was obtained. The mes-trimethoxybenzene (83.89 mg, 0.5 mmol) was used as the internal standard. The stock solution was dissolved in CDCl₃ stirring for 10 minutes, sampled, and equipped with nuclear magnetism. After calculation, conversion rate of 1H is 99%. 1H NMR analysis of the product is: 1H NMR (400 MHz, CDCl₃): δ 7.28 (d, 2H, ArCH), 7.19 (d, 2H, ArCH), 4.82 (s, 2H, OCH2), 1.23 (s, 9H, CH3, tBu), 1.18 (s, 36H, CH3). The boric acid ester was further hydrolyzed into alcohol, add 1 g silica gel with 3 mL methanol as the solvent at 50° C. for 3 h. After the reaction, the product pure primary alcohol was extracted three times with ethyl acetate, and the organic layers were combined, dried with anhydrous sodium sulfate, the solvent was removed under reduced pressure, and purified by silica gel (100-200 mesh) column chromatography, washed with ethyl acetate/hexane (1:5 volume ratio) mixture, with a separation yield of 92%. 1H NMR analysis of the product is: 1H NMR (400 MHz, CDCl₃): δ 7.28 (d, 2H, ArCH), 7.19 (d, 2H, ArCH), 4.51 (s, 2H, CH2), 2.12 (br s, 1H, OH), 1.23 (s, 9H, CH3, tBu).

### Example 10: Borate Ester Prepared from Pinacolborane and 2-Bromobenzoic Acid with a Molar Ratio of 4:1

Under the protection of N₂, loading the 2-bromobenzoic acid (100.6 mg, 0.5 mmol) to the reaction bottle, which is treated by dehydration and deoxidation, then adding a pinacolborane (290 μL, 2 mmol) with pipette gun, after reacting for 12 h at room temperature, the reaction was removed from the glove box, the reaction was terminated, and the borate was obtained. The mes-trimethoxybenzene (84.17 mg, 0.5 mmol) was used as the internal standard. The stock solution was dissolved in CDCl₃ stirring for 10 minutes, sampled, and equipped with nuclear magnetism. After calculation, conversion rate of 1H is 99%. 1H NMR analysis of the product is: 1H NMR (400 MHz, CDCl₃): δ 7.41 (d, 2H, ArCH), 7.19-7.22 (m, 1H, ArCH), 7.03 (t, 1H, ArCH), 4.90 (s, 2H, OCH2), 1.19 (s, 36H, CH3). The boric acid ester was further hydrolyzed into alcohol, add 1 g silica gel with 3 mL methanol as the solvent at 50° C. for 3 h. After the reaction, the product pure primary alcohol was extracted three times with ethyl acetate, and the organic layers were combined, dried with anhydrous sodium sulfate, the solvent was removed under reduced pressure, and purified by silica gel (100-200 mesh) column chromatography, washed with ethyl acetate/hexane (1:5 volume ratio) mixture, with a separation yield of 92%. 1H NMR analysis of the product is: 1H NMR (400 MHz, CDCl₃): δ 7.41 (d, 2H, ArCH), 7.19-7.22 (m, 1H, ArCH), 7.03 (t, 1H, ArCH), 4.71 (s, 2H, CH2), 2.41 (br s, 1H, OH).

### Example 11: Borate Ester Prepared from Pinacolborane and 4-Iodobenzoic Acid with a Molar Ratio of 4:1

Under the protection of N₂, loading the 4-iodobenzoic acid (124.0 mg, 0.5 mmol) to the reaction bottle, which is treated by dehydration and deoxidation, then adding a pinacolborane (290 μL, 2 mmol) with pipette gun, after reacting for 12 h at room temperature, the reaction was removed from the glove box, the reaction was terminated, and the borate was obtained. The mes-trimethoxybenzene (84.09 mg, 0.5 mmol) was used as the internal standard. The stock solution was dissolved in CDCl₃ stirring for 10 minutes, sampled, and equipped with nuclear magnetism. After calculation, conversion rate of 1H is 99%. 1H NMR analysis of the product is: 1H NMR (400 MHz, CDCl₃): δ 7.57 (d, 2H, ArCH), 7.02 (d, 2H, ArCH), 4.78 (s, 2H, OCH2), 1.18 (s, 36H, CH3). The boric acid ester was further hydrolyzed into alcohol, add 1 g silica gel with 3 mL methanol as the solvent at 50° C. for 3 h. After the reaction, the product pure primary alcohol was extracted three times with ethyl acetate, and the organic layers were combined, dried with anhydrous sodium sulfate, the solvent was removed under reduced pressure, and purified by silica gel (100-200 mesh) column chromatography, washed with ethyl acetate/hexane (1:5 volume ratio) mixture, with a separation yield of 94%. 1H NMR analysis of the product is: 1H NMR (400 MHz, CDCl₃): δ 7.57 (d, 2H, ArCH), 7.02 (d, 2H, ArCH), 4.65 (s, 2H, CH2), 2.151 (br s, 1H, OH).

### Example 12: Borate Ester Prepared from Pinacolborane and 3-Phenylpropionic Acid with a Molar Ratio of 4:1

Under the protection of N₂, loading the 3-phenylpropionic acid (74.9 mg, 0.2 mmol) to the reaction bottle, which is treated by dehydration and deoxidation, then adding a pinacolborane (289 μL, 2 mmol) with pipette gun, after reacting for 10 h at room temperature, the reaction was removed from the glove box, the reaction was terminated, and the borate was obtained. The mes-trimethoxybenzene (83.89 mg, 0.5 mmol) was used as the internal standard. The stock solution was dissolved in CDCl3 stirring for 10 minutes, sampled, and equipped with nuclear magnetism. After calculation, conversion rate of 1H is 99%. 1H NMR analysis of the product is: 1H NMR (400 MHz, CDCl₃): δ 7.18 (t, 2H, ArCH), 7.05-7.10 (m, 3H, ArCH), 3.80 (t, 2H, CH2, OCH2), 2.62 (t, 2H, CH2), 1.76-1.83 (m, 2H, CH2), 1.17 (s, 36H, CH3). The boric acid ester was further hydrolyzed into alcohol, add 1 g silica gel with 3 mL methanol as the solvent at 50° C. for 3 h. After the reaction, the product pure primary alcohol was extracted three times with ethyl acetate, and the organic layers were combined, dried with anhydrous sodium sulfate, the solvent was removed under reduced pressure, and purified by silica gel (100-200 mesh) column chromatography, washed with ethyl acetate/hexane (1:5 volume ratio) mixture, with a separation yield of 93%. 1H NMR analysis of the product is: 1H NMR (400 MHz, CDCl₃): δ 7.23 (t, 2H, ArCH), 7.11-7.13 (m, 3H, ArCH), 3.60 (t, 2H, CH2, OCH2), 2.65 (t, 2H, CH2), 1.78-1.85 (m, 2H, CH2), 1.61 (br s, 1H, OH).

### Example 13: Borate Ester Prepared from Pinacolborane and Diphenylacetic Acid with a Molar Ratio of 4:1

Under the protection of N₂, loading the diphenylacetic acid (105.8 mg, 0.5 mmol) to the reaction bottle, which is treated by dehydration and deoxidation, then adding a pinacolborane (289 μL, 2 mmol) with pipette gun, after reacting for 12 h at room temperature, the reaction was removed from the glove box, the reaction was terminated, and the borate was obtained. The mes-trimethoxybenzene (83.84 mg, 0.5 mmol) was used as the internal standard. The stock solution was dissolved in CDCl₃ stirring for 10 minutes, sampled, and equipped with nuclear magnetism. After calculation, conversion rate of 1H is 99%. 1H NMR analysis of the product is: 1H NMR (400 MHz, CDCl₃): δ 7.14-7.33 (m, 10H, ArCH), 4.42 (d, 2H, CH2, OCH2), 4.25 (t, 1H, CH), 1.24 (s, 24H, CH3, pinBOBpin), 1.13 (s, 12H, CH3, OBpin). The boric acid ester was further hydrolyzed into alcohol, add 1 g silica gel with 3 mL methanol as the solvent at 50° C. for 3 h. After the reaction, the product pure primary alcohol was extracted three times with ethyl acetate, and the organic layers were combined, dried with anhydrous sodium sulfate, the solvent was removed under reduced pressure, and purified by silica gel (100-200 mesh) column chromatography, washed with ethyl acetate/hexane (1:5 volume ratio) mixture, with a separation yield of 92%. 1H NMR analysis of the product is: 1H NMR (400 MHz, CDCl3): δ 7.20-7.31 (m, 10H, ArCH), 4.19 (t, 1H, CH), 4.13 (d, 2H, CH2), 1.64-1.70 (t, 1H, OH).

### Example 14: Borate Ester Prepared from Pinacolborane and 2-Methyl 5-Bromobenzoic Acid with a Molar Ratio of 4:1

Under the protection of N2, loading the 2-methyl 5-Bromobenzoic acid (107.1 mg, 0.5 mmol) to the reaction bottle, which is treated by dehydration and deoxidation, then adding a pinacolborane (289 μL, 2 mmol) with pipette gun, after reacting for 12 h at room temperature, the reaction was removed from the glove box, the reaction was terminated, and the borate was obtained. The mes-trimethoxybenzene (83.77 mg, 0.5 mmol) was used as the internal standard. The stock solution was dissolved in CDCl3 stirring for 10 minutes, sampled, and equipped with nuclear magnetism. After calculation, conversion rate of 1H is 99%. 1H NMR analysis of the product is: 1H NMR (400 MHz, CDCl₃): δ 6.91 (d, 1H, ArCH), 7.20 (d, 1H, ArCH), 7.48 (s, 1H, ArCH), 4.78 (s, 2H, OCH2), 2.13 (s, 3H, CH3), 1.18 (s, 36H, CH3). The boric acid ester was further hydrolyzed into alcohol, add 1 g silica gel with 3 mL methanol as the solvent at 50° C. for 3 h. After the reaction, the product pure primary alcohol was extracted three times with ethyl acetate, and the organic layers were combined, dried with anhydrous sodium sulfate, the solvent was removed under reduced pressure, and purified by silica gel (100-200 mesh) column chromatography, washed with ethyl acetate/hexane (1:5 volume ratio) mixture, with a separation yield of 92%. 1H NMR analysis of the product is: 1H NMR (400 MHz, CDCl3): δ 6.91 (d, 1H, ArCH), 7.22 (d, 1H, ArCH), 7.57 (s, 1H, ArCH), 4.44 (s, 2H, OCH2), 2.13 (s, 3H, CH3), 2.25 (br s, 1H, OH).

### Example 15: Borate Ester Prepared from Pinacolborane and 2-Phenylbutyric Acid with a Molar Ratio of 4:1

Under the protection of N₂, loading the 2-phenylbutyric acid (82.2 mg, 0.5 mmol) to the reaction bottle, which is treated by dehydration and deoxidation, then adding a pinacolborane (290 μL, 2 mmol) with pipette gun, after reacting for 12 h at room temperature, the reaction was removed from the glove box, the reaction was terminated, and the borate was obtained. The mes-trimethoxybenzene (84.2 mg, 0.5 mmol) was used as the internal standard. The stock solution was dissolved in CDCl₃ stirring for 10 minutes, sampled, and equipped with nuclear magnetism. After calculation, conversion rate of 1H is 99%. 1H NMR analysis of the product is: 1H NMR (400 MHz, CDCl₃): δ 7.16-7.20 (m, 2H, ArCH), 7.09-7.11 (m, 3H, ArCH), 3.84-3.94 (m, 2H, CH2, OCH2), 2.58-2.67 (m, 1H, CH), 1.71-1.80 (m, 1H, CH2), 1.47-1.56 (m, 1H, CH2), 1.17 (s, 36H, CH3, OBpin & pinBOBpin), 0.75 (t, 3H, CH3). The boric acid ester was further hydrolyzed into alcohol, add 1 g silica gel with 3 mL methanol as the solvent at 50° C. for 3 h. After the reaction, the product pure primary alcohol was extracted three times with ethyl acetate, and the organic layers were combined, dried with anhydrous sodium sulfate, the solvent was removed under reduced pressure, and purified by silica gel (100-200 mesh) column chromatography, washed with ethyl acetate/hexane (1:5 volume ratio) mixture, with a separation yield of 92%. 1H NMR analysis of the product is: 1H NMR (400 MHz, CDCl₃): δ 7.17-7.30 (m, 5H, ArCH), 3.66-3.68 (m, 2H, CH2, OCH2), 2.64 (m, 1H, CH), 1.54-1.73 (m, 1H, CH2), 1.87 (s, 1H, OH), 0.81 (t, 3H, CH3).

### Example 16: Borate Ester Prepared from Pinacolborane and Indole-3-Acetic Acid with a Molar Ratio of 4:1

Under the protection of N₂, loading the indole-3-acetic acid (88 mg, 0.5 mmol) to the reaction bottle, which is treated by dehydration and deoxidation, then adding a pinacolborane (363 μL, 2.5 mmol) with pipette gun, after reacting for 12 h at room temperature, the reaction was removed from the glove box, the reaction was terminated, and the borate was obtained. The mes-trimethoxybenzene (84.49 mg, 0.5 mmol) was used as the internal standard. The stock solution was dissolved in CDCl3 stirring for 10 minutes, sampled, and equipped with nuclear magnetism. After calculation, conversion rate of 1H is 95%. 1H NMR analysis of the product is: 1H NMR (400 MHz, CDCl₃): δ 7.83 (d, 1H, ArCH), 7.46 (d, 1H, ArCH), 7.03-7.15 (m, 3H, ArCH), 4.07 (t, 2H, OCH2), 2.91 (t, 2H, CH2), 1.30 (s, 12H, CH3, N-Bpin) 1.15 (s, 24H, CH3, pinBOBpin), 1.07 (s, 12H, CH3, OBpin). The boric acid ester was further hydrolyzed into alcohol, add 1 g silica gel with 3 mL methanol as the solvent at 50° C. for 3 h. After the reaction, the product pure primary alcohol was extracted three times with ethyl acetate, and the organic layers were combined, dried with anhydrous sodium sulfate, the solvent was removed under reduced pressure, and purified by silica gel (100-200 mesh) column chromatography, washed with ethyl acetate/hexane (1:5 volume ratio) mixture, with a separation yield of 92%. 1H NMR analysis of the product is: 1H NMR (400 MHz, CDCl₃): δ 8.10 (s, 1H, NH), 7.83 (d, 1H, ArCH), 7.46 (d, 1H, ArCH), 7.03-7.15 (m, 3H, ArCH), 4.67 (t, 2H, OCH2), 3.28 (t, 2H, CH2), 1.90 (br s, 1H, OH).

### Example 17: Borate Ester Prepared from Pinacolborane and o-Carboxyphenylacetic Acid with a Molar Ratio of 4:1

Under the protection of N₂, loading the o-carboxyphenylacetic acid (90 mg, 0.5 mmol) to the reaction bottle, which is treated by dehydration and deoxidation, then adding a pinacolborane (508 μL, 3.5 mmol) with pipette gun, after reacting for 11 h at room temperature, the reaction was removed from the glove box, the reaction was terminated, and the borate was obtained. The mes-trimethoxybenzene (84.02 mg, 0.5 mmol) was used as the internal standard. The stock solution was dissolved in CDCl₃ stirring for 10 minutes, sampled, and equipped with nuclear magnetism. After calculation, conversion rate of 1H is 99%. 1H NMR analysis of the product is: 1H NMR (400 MHz, CDCl₃): δ 7.34 (br s, 1H, ArCH), 7.12 (br s, 3H, ArCH), 4.91 (s, 2H, CH2), 3.97 (t, 2H, CH2), 2.87 (t, 2H, CH2), 1.18 (s, 72H, CH3, OBpin & pinBOBpin). The boric acid ester was further hydrolyzed into alcohol, add 1 g silica gel with 3 mL methanol as the solvent at 50° C. for 3 h. After the reaction, the product pure primary alcohol was extracted three times with ethyl acetate, and the organic layers were combined, dried with anhydrous sodium sulfate, the solvent was removed under reduced pressure, and purified by silica gel (100-200 mesh) column chromatography, washed with ethyl acetate/hexane (1:5 volume ratio) mixture, with a separation yield of 92%. 1H NMR analysis of the product is: 1H NMR (400 MHz, CDCl₃): δ 7.34 (br s, 1H, ArCH), 7.12 (br s, 3H, ArCH), 4.54 (s, 2H, CH2), 3.76 (t, 2H, CH2OH), 3.7 (br, 1H, OH), 3.1 (br s, 1H, OH), 2.86 (t, 2H, CH2).

### Example 18: Pinacolborane and Acetic Acid with a Molar Ratio of 4:1

Under the protection of N₂, loading the acetic acid (28.6 μL, 0.5 mmol) to the reaction bottle, which is treated by dehydration and deoxidation, then adding a pinacolborane (290 μL, 2 mmol) with pipette gun, after reacting for 10 h at room temperature, the reaction was removed from the glove box, the reaction was terminated, and the borate was obtained. The mes-trimethoxybenzene (84.02 mg, 0.5 mmol) was used as the internal standard. The stock solution was dissolved in CDCl₃ stirring for 10 minutes, sampled, and equipped with nuclear magnetism. After calculation, conversion rate of 1H is 99%. 1H NMR analysis of the product is: 1H NMR (400 MHz, CDCl₃): δ 3.89 (q, 2H, CH2), 1.26 (s, 36H, CH3), 1.22 (br s, 3H, CH3). The boric acid ester was further hydrolyzed into alcohol, add 1 g silica gel with 3 mL methanol as the solvent at 50° C. for 3 h. After the reaction, the product pure primary alcohol was extracted three times with ethyl acetate, and the organic layers were combined, dried with anhydrous sodium sulfate, the solvent was removed under reduced pressure, and purified by silica gel (100-200 mesh) column chromatography, washed with ethyl acetate/hexane (1:5 volume ratio) mixture, with a separation yield of 92%. For the reaction diagram, see the FIG. 2, R is from acetic acid. 1H NMR analysis of the product is: 1H NMR (400 MHz, CDCl₃): δ 3.69 (q, 2H, CH2), 2.92 (br s, 1H, OH), 1.22 (br s, 3H, CH3).

### Example 19: Pinacolborane and Valeric Acid with a Molar Ratio of 4:1

Under the protection of N₂, loading the valeric acid (54.38 μL, 0.5 mmol) to the reaction bottle, which is treated by dehydration and deoxidation, then adding a pinacolborane (290 μL, 2 mmol) with pipette gun, after reacting for 10 h at room temperature, the reaction was removed from the glove box, the reaction was terminated, and the borate was obtained. The mes-trimethoxybenzene (84.12 mg, 0.5 mmol) was used as the internal standard. The stock solution was dissolved in CDCl₃ stirring for 10 minutes, sampled, and equipped with nuclear magnetism. After calculation, conversion rate of 1H is 92%. 1H NMR analysis of the product is: 1H NMR (400 MHz, CDCl₃): δ 3.82 (t, 2H, OCH2), 1.53-1.57 (m, 2H, CH2), 1.31-1.53 (m, 4H, CH2), 1.29 (s, 36H, CH), 0.87 (t, 3H, CH3). The boric acid ester was further hydrolyzed into alcohol, add 1 g silica gel with 3 mL methanol as the solvent at 50° C. for 3 h. After the reaction, the product pure primary alcohol was extracted three times with ethyl acetate, and the organic layers were combined, dried with anhydrous sodium sulfate, the solvent was removed under reduced pressure, and purified by silica gel (100-200 mesh) column chromatography, washed with ethyl acetate/hexane (1:5 volume ratio) mixture, with a separation yield of 87%. 1H NMR analysis of the product is: 1H NMR (400 MHz, CDCl₃): δ 3.63 (t, 2H, OCH2), 1.57 (m, 2H, CH2), 1.35 (m, 2H, CH2), 2.35 (br s, 1H, OH), 0.91 (t, 3H, CH3).

### Example 20: Pinacolborane and Caproic Acid with a Molar Ratio of 4:1

Under the protection of N₂, loading the caproic acid (62.52 μL, 0.5 mmol) to the reaction bottle, which is treated by dehydration and deoxidation, then adding a pinacolborane (290 μL, 2 mmol) with pipette gun, after reacting for 10 h at room temperature, the reaction was removed from the glove box, the reaction was terminated, and the borate was obtained. The mes-trimethoxybenzene (84.01 mg, 0.5 mmol) was used as the internal standard. The stock solution was dissolved in CDCl₃ stirring for 10 minutes, sampled, and equipped with nuclear magnetism. After calculation, conversion rate of 1H is 90%. 1H NMR analysis of the product is: 1H NMR (400 MHz, CDCl₃): δ 3.77 (t, 2H, OCH2), 1.47-1.53 (m, 2H, CH2), 1.25-1.36 (m, 6H, CH2), 1.2 (s, 48H, CH3), 0.83 (t, 3H, CH3). The boric acid ester was further hydrolyzed into alcohol, add 1 g silica gel with 3 mL methanol as the solvent at 50° C. for 3 h. After the reaction, the product pure primary alcohol was extracted three times with ethyl acetate, and the organic layers were combined, dried with anhydrous sodium sulfate, the solvent was removed under reduced pressure, and purified by silica gel (100-200 mesh) column chromatography, washed with ethyl acetate/hexane (1:5 volume ratio) mixture, with a separation yield of 83%. 1H NMR analysis of the product is: 1H NMR (400 MHz, CDCl3): δ 3.75 (t, 2H, OCH2), 1.47-1.53 (m, 2H, CH2), 1.25-1.36 (m, 6H, CH2), 1.72 (br s, 1H, OH), 0.83 (t, 3H, CH3).

### Example 21: Pinacolborane and Heptanoic Acid with a Molar Ratio of 4:1

Under the protection of N₂, loading the heptanoic acid (70.90 μL, 0.5 mmol) to the reaction bottle, which is treated by dehydration and deoxidation, then adding a pinacolborane (290 μL, 2 mmol) with pipette gun, after reacting for 10 h at room temperature, the reaction was removed from the glove box, the reaction was terminated, and the borate was obtained. The mes-trimethoxybenzene (84.05 mg, 0.5 mmol) was used as the internal standard. The stock solution was dissolved in CDCl₃ stirring for 10 minutes, sampled, and equipped with nuclear magnetism. After calculation, conversion rate of 1H is 90%. 1H NMR analysis of the product is: 1H NMR (400 MHz, CDCl₃): δ 3.72 (t, 2H, OCH2), 1.42-1.48 (m, 2H, CH2), 1.20-1.31 (m, 8H, CH2), 1.15 (s, 48H, CH3), 0.78 (t, 3H, CH3). The boric acid ester was further hydrolyzed into alcohol, add 1 g silica gel with 3 mL methanol as the solvent at 50° C. for 3 h. After the reaction, the product pure primary alcohol was extracted three times with ethyl acetate, and the organic layers were combined, dried with anhydrous sodium sulfate, the solvent was removed under reduced pressure, and purified by silica gel (100-200 mesh) column chromatography, washed with ethyl acetate/hexane (1:5 volume ratio) mixture, with a separation yield of 82%. 1H NMR analysis of the product is: 1H NMR (400 MHz, CDCl₃): δ 3.72 (t, 2H, OCH2), 1.42-1.48 (m, 2H, CH2), 1.20-1.31 (m, 8H, CH2), 1.75 (br s, 1H, OH), 0.78 (t, 3H, CH3).

### Example 22: Pinacolborane and Trimethylacetic Acid with a Molar Ratio of 4:1

Under the protection of N₂, loading the trimethylacetic acid (50.7 mg, 0.5 mmol) to the reaction bottle, which is treated by dehydration and deoxidation, then adding a pinacolborane (289 μL, 2 mmol) with pipette gun, after reacting for 10 h at room temperature, the reaction was removed from the glove box, the reaction was terminated, and the borate was obtained. The mes-trimethoxybenzene (84.08 mg, 0.5 mmol) was used as the internal standard. The stock solution was dissolved in CDCl₃ stirring for 10 minutes, sampled, and equipped with nuclear magnetism. After calculation, conversion rate of 1H is 99%. 1H NMR analysis of the product is: 1H NMR (400 MHz, CDCl₃): δ 3.44 (s, 2H, OCH2), 1.18 (s, 36H, CH3, OBpin & pinBOBpin), 0.83 (s, 9H, CH3). The boric acid ester was further hydrolyzed into alcohol, add 1 g silica gel with 3 mL methanol as the solvent at 50° C. for 3 h. After the reaction, the product pure primary alcohol was extracted three times with ethyl acetate, and the organic layers were combined, dried with anhydrous sodium sulfate, the solvent was removed under reduced pressure, and purified by silica gel (100-200 mesh) column chromatography, washed with ethyl acetate/hexane (1:5 volume ratio) mixture, with a separation yield of 90%. 1H NMR analysis of the product is: 1H NMR (400 MHz, CDCl₃): δ 3.20 (s, 2H, OCH2), 1.97 (br s, 1H, OH), 0.83 (s, 9H, CH3).

### Example 23: Pinacolborane and Adipic Acid with a Molar Ratio of 7:1

Under the protection of N₂, loading the adipic acid (72.9 mg, 0.5 mmol) to the reaction bottle, which is treated by dehydration and deoxidation, then adding a pinacolborane (508 μL, 3.5 mmol) with pipette gun, after reacting for 10 h at room temperature, the reaction was removed from the glove box, the reaction was terminated, and the borate was obtained. The mes-trimethoxybenzene (84.08 mg, 0.5 mmol) was used as the internal standard. The stock solution was dissolved in CDCl₃ stirring for 10 minutes, sampled, and equipped with nuclear magnetism. After calculation, conversion rate of 1H is 99%. 1H NMR analysis of the product is: 1H NMR (400 MHz, CDCl₃): δ 3.78 (t, 4H, OCH2), 1.49-1.51 (m, 4H, CH2), 1.29-1.31 (m, 4H, CH2), 1.18 (s, 72H, CH3, OBpin & pinBOBpin). The boric acid ester was further hydrolyzed into alcohol, add 1 g silica gel with 3 mL methanol as the solvent at 50° C. for 3 h. After the reaction, the product pure primary alcohol was extracted three times with ethyl acetate, and the organic layers were combined, dried with anhydrous sodium sulfate, the solvent was removed under reduced pressure, and purified by silica gel (100-200 mesh) column chromatography, washed with ethyl acetate/hexane (1:5 volume ratio) mixture, with a separation yield of 91%. 1H NMR analysis of the product is: 1H NMR (400 MHz, CDCl₃): δ 3.75 (t, 4H, OCH2), 1.48-1.51 (m, 4H, CH2), 1.29-1.31 (m, 4H, CH2), 2.26 (br s, 2H, OH).

### Example 24: Pinacolborane and Acetic Acid with a Molar Ratio of 3:1

Under the protection of N₂, loading the acetic acid (28.6 μL, 0.5 mmol) to the reaction bottle, which is treated by dehydration and deoxidation, then adding a pinacolborane (218 μL, 1.5 mmol) with pipette gun, after reacting for 10 h at room temperature, the reaction was removed from the glove box, the reaction was terminated, and the borate was obtained. The mes-trimethoxybenzene (84.08 mg, 0.5 mmol) was used as the internal standard. The stock solution was dissolved in CDCl₃ stirring for 10 minutes, sampled, and equipped with nuclear magnetism. After calculation, conversion rate of 1H is 95%. 1H NMR analysis of the product is: 1H NMR (400 MHz, CDCl₃): δ 3.89 (q, 2H, CH2), 1.26 (s, 36H, CH3), 1.22 (br s, 3H, CH3). The boric acid ester was further hydrolyzed into alcohol, add 1 g silica gel with 3 mL methanol as the solvent at 50° C. for 3 h. After the reaction, the product pure primary alcohol was extracted three times with ethyl acetate, and the organic layers were combined, dried with anhydrous sodium sulfate, the solvent was removed under reduced pressure, and purified by silica gel (100-200 mesh) column chromatography, washed with ethyl acetate/hexane (1:5 volume ratio) mixture, with a separation yield of 90%. 1H NMR analysis of the product is: 1H NMR (400 MHz, CDCl₃): δ 3.69 (q, 2H, CH2), 2.92 (br s, 1H₂OH), 1.22 (br s, 3H, CH3).

### Example 25: Pinacolborane and Acetic Acid with a Molar Ratio of 5:1

Under the protection of N₂, loading the acetic acid (28.6 μL, 0.5 mmol) to the reaction bottle, which is treated by dehydration and deoxidation, then adding a pinacolborane (363 μL, 2.5 mmol) with pipette gun, after reacting for 10 h at room temperature, the reaction was removed from the glove box, the reaction was terminated, and the borate was obtained. The mes-trimethoxybenzene (84.08 mg, 0.5 mmol) was used as the internal standard. The stock solution was dissolved in CDCl₃ stirring for 10 minutes, sampled, and equipped with nuclear magnetism. After calculation, conversion rate of 1H was 15%. 1H NMR analysis of the product was: 1H NMR (400 MHz, CDCl₃): δ 3.89 (q, 2H, CH2), 1.26 (s, 36H, CH3), 1.22 (br s, 3H, CH3). The boric acid ester was further hydrolyzed into alcohol, add 1 g silica gel with 3 mL methanol as the solvent at 50° C. for 3 h. After the reaction, the product pure primary alcohol was extracted three times with ethyl acetate, and the organic layers were combined, dried with anhydrous sodium sulfate, the solvent was removed under reduced pressure, and purified by silica gel (100-200 mesh) column chromatography, washed with ethyl acetate/hexane (1:5 volume ratio) mixture, with a separation yield of 92%. 1H NMR analysis of the product is: 1H NMR (400 MHz, CDCl₃): δ 3.69 (q, 2H, CH2), 2.92 (br s, 1H, OH), 1.22 (br s, 3H, CH3).

### Example 26

Under the protection of N₂, loading the benzoic acid (3.66 g, 30 mmol) to the reaction bottle, which is treated by dehydration and deoxidation, then adding a pinacolborane (14.37 mL, 99 mmol) with pipette gun, after reacting for 12 h at room temperature, the reaction was removed from the glove box, the mes-trimethoxybenzene (0.5 mmol) was used as the internal standard. The stock solution was dissolved in CDCl3 stirring for 10 minutes, sampled, and equipped with nuclear magnetism. After calculation, conversion rate of 1H is 99%. 1H NMR analysis of the product is: 1H NMR (400 MHz, CDCl₃): δ 7.22-7.32 (m, 5H, ArH), 4.92 (s, 2H, CH2), 1.26 (s, 36H, CH3). The boric acid ester was further hydrolyzed into alcohol, add 60 g silica gel with 180 mL methanol as the solvent at 50° C. for 3 h. After the reaction, the product pure primary alcohol was extracted three times with ethyl acetate, and the organic layers were combined, dried with anhydrous sodium sulfate, the solvent was removed under reduced pressure, and purified by silica gel (100-200 mesh) column chromatography, washed with ethyl acetate/hexane (1:5 volume ratio) mixture, with a separation yield of 91%. 1H NMR analysis of the product is: 1H NMR (400 MHz, CDCl₃): δ 7.22-7.29 (m, 5H, ArH), 4.61 (s, 2H, CH2), 1.87 (br s, 1H, OH).

### Example 27

Under the protection of N₂, loading the benzoic acid (6.11 g, 50 mmol) to the reaction bottle, which is treated by dehydration and deoxidation, then adding a pinacolborane (23.94 mL, 165 mmol) with pipette gun, after reacting for 12 h at room temperature, the reaction was removed from the glove box, the mes-trimethoxybenzene (0.5 mmol) was used as the internal standard. The stock solution was dissolved in CDCl₃ stirring for 10 minutes, sampled, and equipped with nuclear magnetism. After calculation, conversion rate of 1H is 99%. 1H NMR analysis of the product is: 1H NMR (400 MHz, CDCl₃): δ 7.22-7.32 (m, 5H, ArH), 4.92 (s, 2H, CH2), 1.26 (s, 36H, CH3). The boric acid ester was further hydrolyzed into alcohol, with a separation yield of 91%.

### Example 28

Under the protection of N₂, loading the acetic acid (2.86 mL, 50 mmol) to the reaction bottle, which is treated by dehydration and deoxidation, then adding a pinacolborane (29 mL, 0.2 mol) with pipette gun, after reacting for 10 h at room temperature, the reaction was removed from the glove box, the mes-trimethoxybenzene (0.5 mmol) was used as the internal standard. The stock solution was dissolved in CDCl₃ stirring for 10 minutes, sampled, and equipped with nuclear magnetism. After calculation, conversion rate of 1H is 99%. 1H NMR analysis of the product is: 1H NMR (400 MHz, CDCl₃): δ 3.89 (q, 2H, CH2), 1.26 (s, 36H, CH3), 1.22 (br s, 3H, CH3). The boric acid ester was further hydrolyzed into alcohol, add 100 g silica gel with 250 mL methanol as the solvent at 50° C. for 3 h. After the reaction, the product pure primary alcohol was extracted three times with ethyl acetate, and the organic layers were combined, dried with anhydrous sodium sulfate, the solvent was removed under reduced pressure, and purified by silica gel (100-200 mesh) column chromatography, washed with ethyl acetate/hexane (1:5 volume ratio) mixture, with a separation yield of 92%. 1H NMR analysis of the product is: 1H NMR (400 MHz, CDCl₃): δ 3.69 (q, 2H, CH2), 2.92 (br s, 1H, OH), 1.22 (br s, 3H, CH3).

The reaction in the embodiment of the present invention is carried out in a glove box; carboxylic acids are generally solid, aliphatic carboxylic acids are generally liquid, and the reaction between carboxylic acid and pinacol borane is a heterogeneous reaction. The reaction of alcohol borane is a homogeneous reaction. The invention is the hydroboration reaction of carboxylic acid without solvent and catalyst, which conforms to the principle of green chemistry.

1. A method for preparing a borate ester by non-catalytic hydroboration of a carboxylic acid, comprising the following steps: without a solvent and a catalyst, conducting a non-catalytic hydroboration of the carboxylic acid and a borane to prepare the borate ester.

2. The method according to claim 1, wherein the borane is pinacolborane; and the carboxylic acid is acetic acid, caproic acid, valeric acid, heptanoic acid, trimethylacetic acid, adipic acid, benzoic acid, 4-bromo-benzoic acid, 4-fluorobenzoic acid, 1-naphthoic acid, 2-methoxybenzoic acid, 4-tert-butylbenzoic acid, 4-ethyl oxybenzoic acid, 2-bromobenzoic acid, 4-iodobenzoic acid, 3-phenylpropionic acid, diphenylacetic acid, 2-phenylbutyric acid, indole-3-acetic acid, o-carboxyphenylacetic acid, or 2-methyl-5-bromobenzoic acid.

3. The method according to claim 1, wherein a molar ratio of the carboxylic acid and borane is from 1:3 to 1:7; and the hydroboration is carried at room temperature for 6 to 12 hours.

4. The method according to claim 1, wherein the hydroboration is carried out under the inert gas atmosphere; and the hydroboration is stopped by contacting air to obtain the borate ester.

5. A method for preparing an alcohol compound from a carboxylic acid, comprising the following steps: without a solvent and a catalyst, conducting a non-catalytic hydroboration of the carboxylic acid and a borane; after the hydroboration, adding silica gel and methanol, conducting a hydrolysis reaction to prepare the alcohol compound.

6. The method according to claim 5, wherein a molar ratio of the carboxylic acid and borane is from 1:3 to 1:7; the borane is pinacolborane; and the carboxylic acid is acetic acid, caproic acid, valeric acid, heptanoic acid, trimethylacetic acid, adipic acid, benzoic acid, 4-bromobenzoic acid, 4-fluorobenzoic acid, 1-naphthoic acid, 2-methoxybenzene Formic acid, 4-tert-butylbenzoic acid, 4-ethoxybenzoic acid, 2-bromobenzoic acid, 4-iodobenzoic acid, 3-phenylpropionic acid, diphenylacetic acid, 2-phenylbutyric acid, indole-3-acetic acid, o-carboxyphenylacetic acid, or 2-methyl-5-bromobenzoic acid.

7. The method according to claim 5, wherein the hydroboration is carried at room temperature for 6 to 12 hours; and the hydrolysis reaction is conducted at 50° C. for 3 h.

8. The method according to claim 5, wherein the hydroboration is under the inert gas atmosphere; the hydroboration is stopped by contacting air, and silica gel and methanol are added to conduct the hydrolysis reaction to prepare the alcohol compound.

9. An application of a carboxylic acid and a borane in preparing a borate ester, wherein the application is carried out without a catalyst; the borane is pinacolborane; and the carboxylic acid is acetic acid, caproic acid, valeric acid, heptanoic acid, trimethylacetic acid, adipic acid, benzoic acid, 4-bromobenzoic acid, 4-fluorobenzoic acid, 1-naphthoic acid, 2-methoxybenzoic acid, 4-tert-butyl benzoic acid, 4-ethoxybenzoic acid, 2-bromobenzoic acid, 4-iodobenzoic acid, 3-phenylpropionic acid, diphenylacetic acid, 2-phenylbutyric acid, indole-3-acetic acid, o-carboxyphenylacetic acid or 2-methyl-5-bromobenzoic acid.

10. The application according to claim 9, wherein a molar ratio of the carboxylic acid and the borane is from 1:3 to 1:7.
